(19) United States
(12) Patent Application Publication
(10) Pub. No.: US 2025/0125052 A1
(43) Pub. Date: Apr. 17, 2025

(54) PREDICTING BIOMATERIAL-IMPLANT SURGICAL OUTCOMES

(71) Applicant: UNIVERSITY OF SOUTH CAROLINA, COLUMBIA, SC (US)

(72) Inventors: MELISSA A. MOSS, COLUMBIA, SC (US); MIHYUN L. WAUGH, CAYCE, SC (US); NICHOLAS D. BOLTIN, COLUMBIA, SC (US); LAUREN WOLF, CAMBRIDGE, MA (US); RONNIE HORNER, OMAHA, NE (US); MATTHEW HERMES, SIMPSONVILLE, SC (US); THOMAS WHEELER, GREENVILLE, SC (US); RICHARD GOODWIN, GREENVILLE, SC (US)

(21) Appl. No.: 18/915,993

(22) Filed: Oct. 15, 2024

Related U.S. Application Data
(60) Provisional application No. 63/589,690, filed on Oct. 12, 2023.

(51) Int. Cl. G16H 50/20; A61B 5/00; G06T 7/00; G16H 50/70

(52) U.S. Cl. CPC G16H 50/20; A61B 5/4318; G06T 7/0012; G16H 50/70; G06T 2207/20081

(57) ABSTRACT

In general, the present disclosure is directed to systems and methods of evaluating a subject's risk of one or more complications associated with pelvic organ prolapse surgery. The method comprising: obtaining, by a computing system comprising one or more computing devices, sample data associated with the subject; obtaining, by a computing system comprising one or more computing devices, medical record data associated with the subject; inputting, by the computing system, the sample data into a machine-learned surgical model; receiving, by the computing system as an output of the machine-learned surgical model, one or more predictions of post-surgical complications of mesh exposure through a vaginal wall associated with the subject; and performing a pelvic organ prolapse repair surgery on the subject.

### CROSS REFERENCE TO RELATED APPLICATION

The present application claims priority to U.S. Provisional Patent Application Ser. No. 63/589,690, having a filing date of Oct. 12, 2023, entitled “Predicting Biomaterial-Implant Surgical Outcomes,” which is incorporated herein by reference in its entirety.

### BACKGROUND

Pelvic organ prolapse (POP) encompasses the protrusion of the pelvic organs into the vaginal wall due to weakened pelvic muscles and increased abdominal pressure. This widespread condition affects about 50% of parous women and 6% of non-parous women between the ages of 20 and 59. Standard treatment has included surgical intervention, which can be augmented via implantation of polypropylene vaginal mesh to provide mechanical support and reinforcement of the prolapsed organ. However, surgical procedures involving the use of polypropylene mesh have demonstrated complications via mesh exposure into the vaginal wall. Unfortunately, post-surgical complications, predominantly mesh exposure into the vaginal wall, lead to additional medical costs, surgical risks, and emotional distress. The current landscape provides no standard preoperative method to predict vaginal mesh exposure following surgical intervention. Consequently, patients can be left with residual symptoms and emotional distress. Some patients elect for surgical reintervention to revise or remove the mesh implantation.

As such, there exists a need in the art for methods and systems to accurately predict pelvic organ prolapse surgical outcomes based on a combination of biological sample data and medical record data.

### SUMMARY

In general, the present disclosure is directed to methods of evaluating a subject's risk of one or more complications associated with pelvic organ prolapse surgery. The method comprises: obtaining, by a computing system comprising one or more computing devices, sample data associated with the subject; obtaining, by a computing system comprising one or more computing devices, medical record data associated with the subject; inputting, by the computing system, the sample data into a machine-learned surgical model; receiving, by the computing system as an output of the machine-learned surgical model, one or more predictions of post-surgical complications of mesh exposure through a vaginal wall associated with the subject; and performing a pelvic organ prolapse repair surgery on the subject, wherein the surgery is performed based at least in part on the one or more predictions of post-surgical complications by the machine-learned surgical model associated with a likelihood of success.

Also, the present disclosure is directed to a computing system. The computing system comprises: a machine-learned surgical model trained with training data; one or more processors; and one or more non-transitory computer-readable media that store instructions that, when executed by the one or more processors, cause the one or more processors to perform operations. According to the present disclosure, the operations comprising: obtaining sample data associated with a subject, wherein the data comprises at least one set of sample data associated with the subject; obtaining medical record data associated with the subject; inputting the sample data into the machine-learned surgical model; receiving, as an output of the machine-learned surgical model, one or more predicted surgical outcomes associated with the subject.

These and other features and aspects, embodiments and advantages of the present invention will become better understood with reference to the following description and appended claims.

### BRIEF DESCRIPTION OF THE DRAWINGS

A full and enabling disclosure of the present disclosure is set forth more particularly in the remainder of the specification, including reference to the accompanying figures, in which:

FIG. 1 depicts an example processing workflow for evaluating a subject's risk of post-surgical complications arising from pelvic organ prolapse surgery.

FIG. 2 depicts an example workflow for predicting pelvic organ prolapse surgery outcomes based on integration of blood cytokine levels and medical record data.

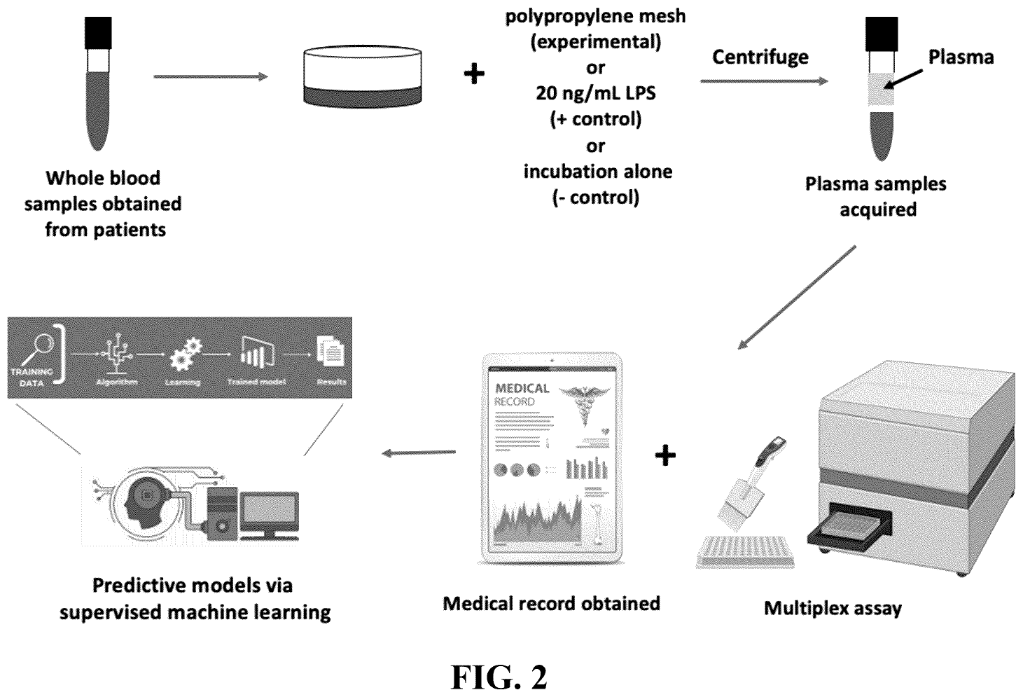

FIG. 3 depicts exemplary medical record data.

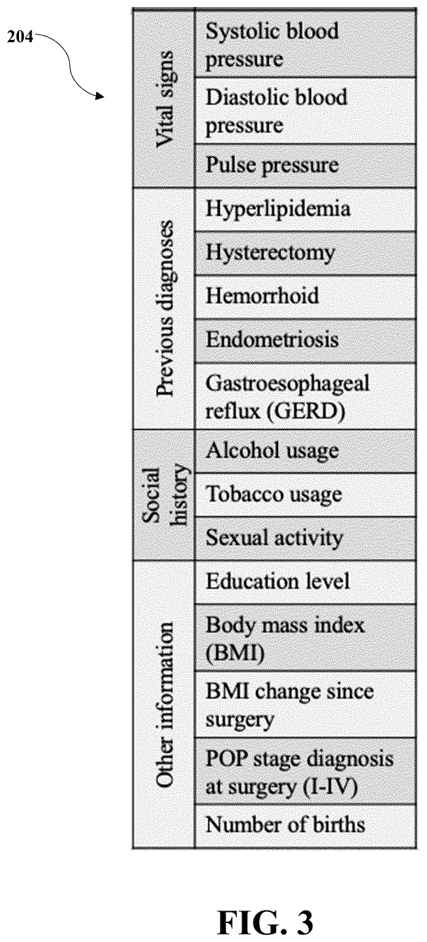

FIG. 4 depicts an example computing system 100 for machine-learning-based analysis of integrated blood cytokine levels and medical record data.

FIG. 5 depicts supervised machine learning models utilized to identify important factors capable of yielding effective predictive analysis: decision tree (DT), Naïve Bayes (NB), logistic regression (LR), and artificial neural network (ANN). Each model was performed with medical data alone, cytokine data alone, or combination of both medical and cytokine data.

Repeat use of reference characters in the present specification and drawings is intended to represent the same or analogous features or elements of the present disclosure.

### DETAILED DESCRIPTION

Reference will now be made in detail to example embodiments of the disclosure. It is to be understood by one of ordinary skill in the art that the present disclosure is a description of exemplary embodiments only and is not intended as limiting the broader aspects of the present disclosure.

The present disclosure is generally directed to systems and methods that include or otherwise leverage a machine-learned surgical model to predict and/or evaluate a subject's risk of post-surgical complications associated with the subject for mesh exposure through the vaginal wall following pelvic organ prolapse surgery. Advantageously, systems and methods disclosed herein utilize supervised learning models with a high degree of accuracy, specificity, and sensitivity that may predict surgical outcomes based on a combination of cytokine level data obtained from a subject's blood sample and a subject's medical record data. For instance, systems and methods disclosed herein may be utilized in a clinical setting for predictive accuracy of post-surgical mesh exposure into the vaginal wall.

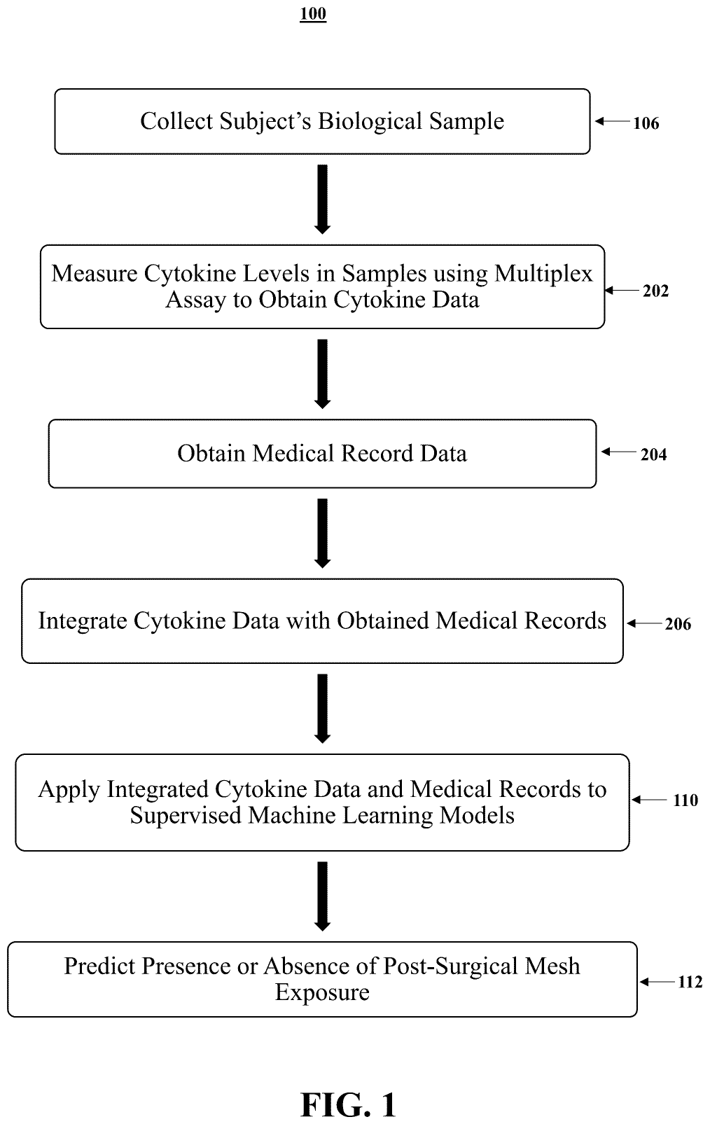

Turning to FIG. 1, in some example embodiments, a biological sample 106 is obtained from a subject. For instance, a clinician may collect and process a biological sample 106 from a subject to classify whether particular surgical markers are expressed at a level in which the subject is likely to have a successful pelvic organ prolapse surgery. In some example embodiments, the present disclosure may be useful for assessing whether or not a pelvic organ prolapse surgery should be performed on a subject based on levels of surgical markers in their biological sample, prior to or following exposure to an inflammatory agent or a biomaterial, using a statistical algorithm and/or empirical data (e.g., the amount of surgical markers described herein, such as in the tables, figures, examples, and otherwise described herein).

In some example embodiments, the biological sample 106 collected from a subject may be a blood sample. In some embodiments, a blood sample may be collected from an upper extremity of a subject. The blood sample may be incubated with inflammatory agent lipopolysaccharide (LPS) or sterile polypropylene mesh. Further, the blood sample from a test subject may be collected and mixed with an agent, such as a protein-binding agent like an antibody or antigen-binding fragment thereof, or a nucleic acid-binding agent like an oligonucleotide, capable of detecting the amount of surgical markers in the biological sample 106. In some embodiments, at least one antibody or antigen-binding fragment thereof is used, wherein two, three, four, five, six, seven, eight, nine, ten, or more such antibodies or antibody fragments can be used in combination (e.g., in multiplex assay) or in serial. In certain instances, the statistical algorithm is a single learning statistical classifier system. For example, a single learning statistical classifier system can be used to classify a sample based on a prediction or probability value and the presence or level of the surgical markers. The use of a single learning statistical classifier system typically classifies the sample as, for example, a likely successful candidate for pelvic organ prolapse repair surgery with a sensitivity, specificity, positive predictive value, negative predictive value, and/or overall accuracy of from about 50% to about 90%, such as at least about 75%, 76%, 77%, 78%, 79%, 80%, 81%, 82%, 83%, 84%, 85%, 86%, 87%, 88%, 89%, or 90%.

In some example embodiments, the subject's biological sample data 106 may include surgical marker data based on the blood sample collected from a subject. According to the present disclosure, levels of surgical markers in each blood sample may be quantified using a multiplex assay 202. According to the present disclosure, surgical markers may include, but are not limited to, pro-inflammatory cytokines, anti-inflammatory cytokines, Treg cytokines, tumor necrosis factor (TNF) superfamily proteins, interferons (IFN) family proteins, T helper 17 (Th17) immunity related genes, and matrix metalloproteinases (MMPs). For instance, surgical markers quantified may include, but are not limited to, IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-12, IL-29/IFN-11, IL-31, IL-32, IL-33, IL-34, IL-35, sIL-6Ra, pg130/sIL-6Rβ, IFN-a2, IFN-β, IFN-γ, TNF-α, sTNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.

In some example embodiments, the subject's biological sample data 106 may be combined with medical record data 204. For instance, the medical record data 204 may be the subject's specific medical record data. In another example embodiment, the medical record data 204 may include medical record data from another subject that has undergone a mesh surgery following a pelvic organ prolapse diagnosis. In some example embodiments, the medical record data 204 may include, but is not limited to, a plurality of the subject's vital sign data, previous diagnoses, social history, and other medical related information.

In one example embodiment, the medical record data 204 may be the subject's vital sign data. For instance, the subject's vital sign data may include, but is not limited to, blood pressure data, pulse pressure data, or the like.

In one example embodiment, the medical record data 204 may be the subject's previous diagnoses data. For instance, previous diagnoses data may include, but is not limited to, hyperlipidemia data, hysterectomy data, hemorrhoid data, endometriosis data, gastroesophageal reflux (GERD) data, or the like.

In one example embodiment, the medical record data 204 may be the subject's social history data. For instance, social history data may include, but is not limited to, alcohol usage data, tobacco usage data, sexual activity data, or the like.

In one example embodiment, the medical record data 204 may be the subject's other medical related information data. For instance, other medical related information data may include, but is not limited to, education level data, body mass index (BMI) data, BMI change since surgery data, pelvic organ prolapse stage diagnosis at surgery (I-IV) data, birth data, or the like.

In some example embodiments, the computing system 100 can input the test data 106 combined with the medical record data 204 into a machine-learned surgical model 110. For example, the machine-learned surgical model 110 may include a machine learning algorithmic technique capable of adapting to complex data sets (e.g., panel of surgical markers of interest) and making decisions based upon such data sets. Examples of the machine-learned surgical model 110 may be learning statistical classifier systems including, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In some embodiments, the machine-learned surgical model 110 may include a deep artificial neural network, a support vector machine, a decision tree, a naïve bayes, and/or a logistic regression.

The machine-learned surgical model 110 and/or associated computing system components may output or otherwise generate and provide a visualization of the one or more cytokine markers expression patterns in the blood sample. Output data may reflect the concentration of cytokine markers expressed in the blood sample. For instance, the output of the machine-learned surgical model 110 can predict post-surgical complications 112 arising from a pelvic organ prolapse repair surgery performed on a subject based on levels of surgical markers and the subject's medical record data. Thus, the model 110 can predict surgical outcome 112 based on levels of surgical markers, thereby providing a treatment guide for clinicians (e.g., surgeons).

Thus, in some implementations, the computing system 100 can take biological sample data 106 combined with medical record data 204 as an input and, in response, provide the clinician a prediction as to whether the post-surgical mesh exposure into the vaginal wall is likely to occur based on predetermined levels of cytokine markers in combination with obtained medical record data. One benefit of the described system may be that the system can guide the treatment expectations for a subject with pelvic organ prolapse prior to surgery.

In some example embodiments, supervised machine learning models disclosed herein may predict the presence or absence of post-surgical mesh exposure. For instance, integrating a subject's cytokine level data with medical record data may be useful in training and testing supervised machine learning models as well as probe the number of cytokine markers required for effective predictions. In some example embodiments, accuracy of the supervised machine learning models may be higher in a model integrated with a subject's cytokine level data and medical record data compared to a model that only includes a subject's cytokine level data. For instance, in one example embodiment, when utilizing combination of both cytokine and medical data to predict the presence or absence of post-surgical mesh exposure, a supervised machine learning model may be more than about 80% accurate, such as more than about 85% accurate, such as more than about 90% accurate, such as more than about 95% accurate.

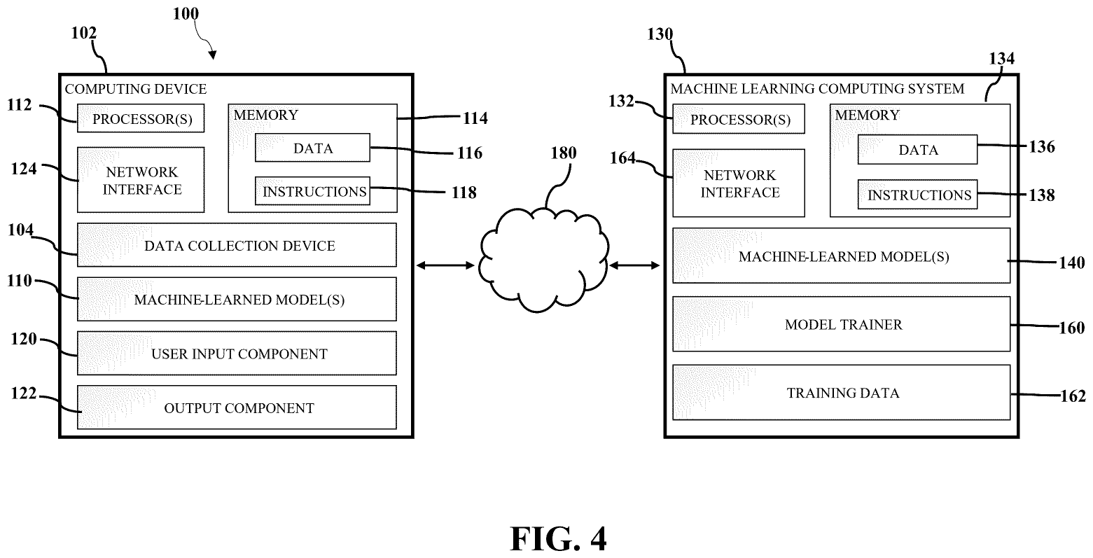

FIG. 4 depicts an example computing system 100 for machine-learning-based analysis of a subject's biological sample data combined with medical record data. The example system 100 includes a computing device 102 and a machine learning computing system 130 that are communicatively coupled over a network 180.

The computing device 102 includes one or more processors 112 and a memory 114. The one or more processors 112 can be any suitable processing device (e.g., a processor core, a microprocessor, an ASIC, an FPGA, a controller, a microcontroller, etc.) and can be one processor or a plurality of processors that are operatively connected. The memory 114 can include one or more non-transitory computer-readable storage media, such as RAM, ROM, EEPROM, EPROM, one or more memory devices, flash memory devices, etc., or a combination thereof.

The memory 114 can store information that can be accessed by the one or more processors 112. For instance, the memory 114 (e.g., one or more non-transitory computer-readable storage mediums, memory devices) can store data 116 that can be obtained, received, accessed, written, manipulated, created, and/or stored. In some implementations, the computing device 102 can obtain data from one or more memory device(s) that are remote from the device 102.

The memory 114 can also store computer-readable instructions 118 that can be executed by the one or more processors 112. The instructions 118 can be software written in any suitable programming language or can be implemented in hardware. Additionally, or alternatively, the instructions 118 can be executed in logically and/or virtually separate threads on processor(s) 112.

For example, the memory 114 can store instructions 118 that when executed by the one or more processors 112 cause the one or more processors 112 to perform any of the operations and/or functions described herein.

According to an aspect of the present disclosure, the computing device 102 can store or include one or more machine-learned models 110. For example, the models 110 can be or can otherwise include various machine-learned models such as a random forest classifier; a logistic regression classifier; a support vector machine; one or more decision trees; a neural network; and/or other types of models including both linear models and non-linear models. Example neural networks include feed-forward neural networks, recurrent neural networks (e.g., long short-term memory recurrent neural networks), convolutional neural networks, or other forms of neural networks.

In some implementations, the computing device 102 can receive the one or more machine-learned models 110 from the machine learning computing system 130 over network 180 and can store the one or more machine-learned models 110 in the memory 114. The computing device 102 can then use or otherwise run the one or more machine-learned models 110 (e.g., by processor(s) 112).

The machine learning computing system 130 includes one or more processors 132 and a memory 134. The one or more processors 132 can be any suitable processing device (e.g., a processor core, a microprocessor, an ASIC, an FPGA, a controller, a microcontroller, etc.) and can be one processor or a plurality of processors that are operatively connected. The memory 134 can include one or more non-transitory computer-readable storage media, such as RAM, ROM, EEPROM, EPROM, one or more memory devices, flash memory devices, etc., or a combination thereof.

The memory 134 can store information that can be accessed by the one or more processors 132. For instance, the memory 134 (e.g., one or more non-transitory computer-readable storage mediums, memory devices) can store data 136 that can be obtained, received, accessed, written, manipulated, created, and/or stored. In some implementations, the machine learning computing system 130 can obtain data from one or more memory device(s) that are remote from the system 130.

The memory 134 can also store computer-readable instructions 138 that can be executed by the one or more processors 132. The instructions 138 can be software written in any suitable programming language or can be implemented in hardware. Additionally, or alternatively, the instructions 138 can be executed in logically and/or virtually separate threads on processor(s) 132.

For example, the memory 134 can store instructions 138 that when executed by the one or more processors 132 cause the one or more processors 132 to perform any of the operations and/or functions described herein.

In some implementations, the machine learning computing system 130 includes one or more server computing devices. If the machine learning computing system 130 includes multiple server computing devices, such server computing devices can operate according to various computing architectures, including, for example, sequential computing architectures, parallel computing architectures, or some combination thereof.

In addition or alternatively to the model(s) 110 at the computing device 102, the machine learning computing system 130 can include one or more machine-learned models 140. For example, the models 140 can be or can otherwise include various machine-learned models such as a random forest classifier; a logistic regression classifier; a support vector machine; one or more decision trees; a neural network; and/or other types of models including both linear models and non-linear models. Example neural networks include feed-forward neural networks, recurrent neural networks (e.g., long short-term memory recurrent neural networks), convolutional neural networks, or other forms of neural networks.

Thus, machine-learned models 110 can be located and used at the computing device 102 and/or machine-learned models 140 can be located and used at the machine learning computing system 130.

In some implementations, the machine learning computing system 130 and/or the computing device 102 can train the machine-learned models 110 and/or 140 through use of a model trainer 160. The model trainer 160 can train the machine-learned models 110 and/or 140 using one or more training or learning algorithms. One example of a training technique is backwards propagation of errors (“backpropagation”).

In some implementations, the model trainer 160 can perform supervised training techniques using a set of labeled training data 162, for example, as described with reference to FIG. 4. In other implementations, the model trainer 160 can perform unsupervised training techniques using a set of unlabeled training data. The model trainer 160 can perform a number of generalization techniques to improve the generalization capability of the models being trained. Generalization techniques include weight decays, dropouts, or other techniques. The model trainer 160 can be implemented in hardware, software, firmware, or combinations thereof.

The computing device 102 can also include a network interface 124 used to communicate with one or more systems or devices, including systems or devices that are remotely located from the computing device 102. The network interface 124 can include any circuits, components, software, etc. for communicating with one or more networks (e.g., 180). In some implementations, the network interface 124 can include, for example, one or more of a communications controller, receiver, transceiver, transmitter, port, conductors, software and/or hardware for communicating data. Similarly, the machine learning computing system 130 can include a network interface 164.

Certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Methods

### Medical Record Data Collection

Medical record data were collected from the 20 female subjects who had experienced prior surgical intervention for POP using transvaginal polypropylene mesh. The data categories include vital signs (systolic blood pressure or systolic BP, diastolic BP, and pulse pressure), previous medical history (hypertension, diabetes, renal disease, hyperlipidemia, hemorrhoids, endometriosis, gastroesophageal reflux, and hysterectomy), social history (alcohol and tobacco usage, sexual activity, marital status, and education level), and other relevant health statistics (age at surgery, body mass index or BMI, BMI change since surgery, POP stage diagnosis at surgery, and number of births). Mean and standard deviation values were calculated for continuous variables (systolic and diastolic BP, pulse pressure, age at surgery, BMI, and BMI changes) along with the range of the variables (Table I). Unpaired t-tests were conducted on each variable to determine significance (p<0.05).

### Sample Collection and Processing

Samples of blood as well as medical records were collected from 20 female patients who had experienced prior surgical intervention for pelvic organ prolapse (POP) using transvaginal polypropylene mesh. Of these subjects, 10 patients experienced exposure of the mesh through the vaginal wall following surgery and 10 had not. Blood samples were incubated with sterile polypropylene mesh (2 cm×2 cm). Plasma layers collected following centrifugation (1500×g, 4° C.) of blood samples were analyzed via multiplex assay to quantify cytokine levels. These cytokines include interleukin-1α (IL 1α), IL-1β, IL 2, IL-4, IL-6, IL-8, IL-10, IL-12 p40, IL-12 p70, IL-17A, interferon-γ (IFN-γ), tumor necrosis factor-α (TNF-α), and granulocyte-macrophage colony-stimulating factor (GM CSF). Three independent measurements were observed to detect the cytokine levels, with each sample evaluated in duplicate. Blood samples from each patient were split into three equal incubation aliquots of:
(1) 20 ng/ml of inflammatory agent lipopolysaccharide (LPS; positive control),
(2) sterile polypropylene mesh (experimental), and
(3) no intervention (negative control).

### Measurement of Blood Cytokine Marker Levels

After 24-h incubation at 37° C., levels of 13 cytokines were measured in the plasma using multiplex assay. As markers of surgical, cytokine levels in each blood sample were quantified using the bead-based MILLIPLEX® Human Cytokine/Chemokine/Growth Factor Panel A—Immunology Multiplex Assay (EMD Millipore Corporation, Billerica, MA), which is comprised of analytes for target markers including, but are not limited to, IL-1α, IL-1β, IL-2, IL-4, IL-6, IL-8, IL-10, IL-12 p40, IL-12 p70, IL-17A, IFN-γ, TNF-α, and GM-CSF. Frozen plasma samples were thawed at room temperature (RT) and analyzed following Milliplex protocol guidelines. Cytokine concentrations were measured using a Bio-Plex® 200 (Bio-Rad, Hercules, CA) and Bio-Plex Manager™ software (Bio-Rad, Hercules, CA). Sample volume was doubled to ensure measurable levels of cytokines, and assay output data was adjusted to reflect concentrations in plasma samples. Each multiplex assay was performed in duplicate, and cytokine levels were evaluated in three independent measurements.

### Descriptive Analytics

The statistical programming language R (version 4.1.2) was used to analyze raw cytokine data values generated from the multiplex immunoassay. The imported data structure contained 60 observations (20 subjects×3 independent measurements) and 40 total variable fields (13 cytokines×3 blood treatments+1 target variable). The target variable was the subject outcome, which indicated post-surgical complication that subjects might have experienced following POP surgery. Observations marked ‘presence’ represent subjects who experienced mesh exposure through the vaginal wall. Observations marked ‘absence’ represent subjects who did not experience any mesh exposure through the vaginal wall. Univariate and multivariate methods were used to explore the data set, including identifying missing values, analyzing outliers, and visualizing frequency distributions.

### Predictive Analytics

After 24-h incubation at 37° C., levels of 13 cytokines were measured in the plasma using multiplex assay. The cytokine data were combined with standardized, patient-specific medical record information. Following integration, 70% and 30% of the data were split, respectively, into training and testing sets for supervised machine learning models. The models were performed under these conditions to predict the presence or absence of post-surgical mesh exposure.

Raw datasets including blood cytokine levels and various medical record data were analyzed using statistical programming language R. The imported data contained 60 observations (20 subjects×3 independent cytokine measurements) and 35 total variable fields (21 medical record variables+13 cytokines+1 target variable). The target variable of exposure referred to the presence or absence of surgical mesh exposure through the vaginal wall following POP surgery. Univariate and multivariate analysis was implemented to explore the dataset, including identifying missing values, analyzing outliers, and standardizing categorical variables.

Supervised machine learning models were created using the caret package (version 6.0-90) in the R programming language. The four models trained were Decision Tree (DT), Logistic Regression (LR), Naive Bayes (NB), and Artificial Neural Network (ANN). This approach focused on three different datasets: (1) patient medical record data, (2) blood cytokine levels following incubation with surgical mesh, and (3) an integrated dataset of both medical record data and blood cytokine levels. Prior to creating the predictive models, the original data were split using an industry standard of 70% for training and 30% for testing. Each group contained an equal distribution of subjects who did or did not experience post-surgical mesh exposure through the vaginal wall, the prediction target for each model. Each model was trained using the 70% (42/60) subset and a cross-validation training control. A 10-fold cross-validation with 25% (15/60) left out replicated three times was used on each model to avoid bias and overfitting. From this, training accuracies were reported. Additional testing was performed for the prediction accuracy of each model using the 30% (18/60) test data. Prediction accuracies are reported along with sensitivity and specificity for the prediction of subjects to experience post-surgical mesh exposure. After computing predictive statistics for each dataset, analysis using the varImp function was conducted to identify which variables contributed most to the predictive outcome within each model. This analysis allowed for visualization of individual variable importance regarding each predictive model.

### Example 1

Healthcare data of POP patients were collected from electronic medical records and analyzed in Table I. The cohort included 20 POP patients, among whom were the 10 that experienced vaginal mesh exposure and 10 that did not. Overall, no significant differences (p<0.05) in systolic and diastolic BP, pulse pressure, age at surgery, BMI, and BMI changes were noted between patients with or without mesh exposure. Higher numbers of patients with no mesh exposure had previous medical diagnosis of hypertension, diabetes, renal diseases, endometriosis, and gastroesophageal reflux, while more patients with mesh exposure had hemorrhoids. In contrast, higher numbers of patients with mesh exposure reported social histories of alcohol and tobacco usage as well as sexual activity.

TABLE I
Demographics of the POP patient study
population (N = 20 subjects).

| | Mesh | No mesh |
|---|---|---|
| | exposure | exposure |
| | (N = 10 | (N = 10 |
| Variables | subjects) | subjects) |
| Vital signs | | |
| Systolic BP | | |
| mean ± SD | 127.4 ± 17.9 | 123.3 ± 19.3 |
| range | 102-162 | 98-152 |
| Diastolic BP | | |
| mean ± SD | 77.9 ± 11.3 | 76.9 ± 10.7 |
| range | 58-98 | 60-92 |
| Pulse pressure | | |
| mean ± SD | 49.5 ± 21.3 | 46.4 ± 11.9 |
| range | 24-104 | 34-64 |
| Previous medical history, N (%) | | |
| Hypertension | 4 (40) | 5 (50) |
| Diabetes | 0 (0) | 2 (20) |
| Renal disease | 2 (20) | 5 (50) |
| Hyperlipidemia | 3 (30) | 3 (30) |
| Hemorrhoids | 3 (30) | 2 (20) |
| Endometriosis | 3 (30) | 4 (40) |
| Gastroesophageal reflux | 5 (50) | 6 (60) |
| Hysterectomy | 8 (80) | 8 (80) |
| Social history, N (%) | | |
| Alcohol | 6 (60) | 2 (20) |
| Tobacco | 2 (20) | 1 (10) |
| Sexual activity | 9 (90) | 5 (50) |
| Marital status | | |
| Single/Lives alone | 0 (0) | 1 (10) |
| Married | 8 (80) | 6 (60) |
| Separated/Divorced | 1 (10) | 1 (10) |
| Widowed | 1 (10) | 2 (20) |
| Highest education level | | |
| Primary | 0 (0) | 1 (10) |
| Secondary | 2 (20) | 2 (20) |
| College or higher | 8 (80) | 7 (70) |
| Other relevant health statistics | | |
| Age at surgery, years | | |
| mean ± SD | 56.8 ± 1.2 | 59.4 ± 8.0 |
| range | 43-77 | 47-75 |
| BMI | | |
| mean ± SD | 29.3 ± 7.0 | 28.8 ± 6.5 |
| range | 19.3-42.3 | 21.5-44.9 |
| BMI change | | |
| mean ± SD | −1.6 ± 4.5 | −1.1 ± 3.5 |
| POP stage pre-op, N (%) | | |
| 1 | 1 (10) | 1 (10) |
| 2 | 4 (40) | 2 (20) |
| 3 | 4 (40) | 7 (70) |
| 4 | 1 (10) | 0 (0) |
| Number of births, N (%) | | |
| 1-2 | 6 (60) | 5 (50) |
| 3+ | 4 (40) | 5 (50) |

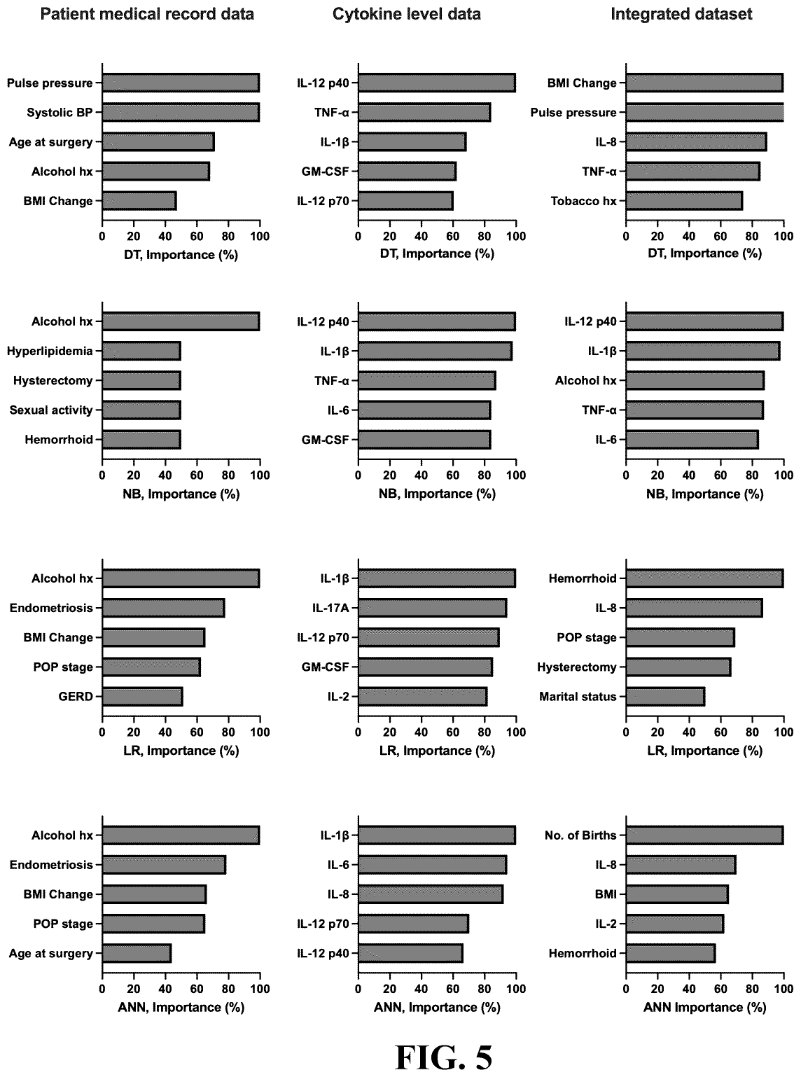

To examine predictive capabilities of cytokine level measurements in supervised machine learning models. Each of four supervised machine learning models, DT, NB, LR, and ANN, were implemented on three separate datasets: subject's medical record data (21 variables), blood cytokine levels following incubation with surgical mesh (13 variables), and an integrated dataset of both medical data and blood cytokine levels (34 variables). The five most important factors that predict mesh exposure in each model are presented in FIG. 5.

When the models were trained with subject's medical record data alone, history of alcohol usage was the most important factor for NB, LR, and ANN models and also exhibited greater than 60% importance in the DT model. BMI change was among the five most relevant factors in three of the models, DT, LR, and ANN. Three additional factors were among the five most relevant factors in two of the four models: medical history of endometriosis (LR and ANN, >77% importance), POP stage (LR and ANN, >60% importance), and age at surgery (DT and ANN, >40% importance).

When models were trained with blood cytokine levels alone, IL-1β was the most important cytokine to predict mesh exposure in LR and ANN models and among the five most important cytokines in NB and DT models, while IL-12 p40 was the most important cytokine in DT and NB models and among the five most important cytokines in the ANN model. Two cytokines exhibited >60% importance in three models: GM-CSF in DT, NB, and LR models and IL-12 p70 in DT, LR, and ANN models. Two cytokines exhibited >80% in two models: TNF-α in DT and NB models and IL-6 in NB and ANN models.

Finally, the predictive models were also performed on an integrated dataset of both medical record data and blood cytokine levels. IL-8 was among the five most important factors (>70% importance) in three models, DT, LR, and ANN. Two factors were among the five most important in two models: TNF-α in DT and NB models and hemorrhoids in LR and ANN models. Factors of highest importance in all four models included a combination of healthcare characteristics and blood cytokine levels. For the NB model, the five most important factors in the integrated dataset matched those identified to be of highest importance in the individual datasets. In the DT model, three factors identified to be of high importance in individual datasets were also of highest importance in the integrated dataset, while two previously unidentified factors rose to high importance within the integrated dataset. In contrast, ANN and LR models identified four unique factors to be of highest importance within the integrated dataset.

Statistics were generated for the performance of each supervised machine learning model (Table II). Each model achieved the highest prediction accuracy when utilizing the integrated dataset, ranging from 78% (14/18) to 94% (17/18), compared to either medical record data or blood cytokine levels alone, which scored ranges of 33% (6/18) to 50% (9/18) and 50% (9/18) to 83% (15/18), respectively. Similarly, the highest training accuracy for each model was observed when using the integrated dataset, as opposed to either medical record data or blood cytokine levels alone. When comparing different models, both LR and ANN achieved the highest training and prediction accuracies with the integrated dataset. In fact, when the LR and ANN models were trained with the integrated dataset to predict mesh exposure, a training accuracy of 91% (38/42) and a prediction accuracy of 94% (17/18) were observed in combination with sensitivity and specificity ranging from 89% (16/18) to 100% (18/18).

TABLE II
Summary of supervised machine learning model statistics. Subject's medical record data
(Med), blood cytokine levels following incubation with surgical mesh (Cyto), and an integrated
dataset of both patient medical record data and blood cytokine levels (Int) were utilized
to predict presence or absence of POP post-surgical mesh exposure. 70% (42/60) of observations
were used for training, and 30% (18/60) of observations were used for testing.

| | | Training | Prediction | | | | | | | | |
|---|---|---|---|---|---|---|---|---|---|---|---|
| | Data | Accuracy | Accuracy | | | | Sensitivity | | Specificity | | Prediction |
| Model | Set | n (%) | n (%) | | 95% CI | | n (%) | | n (%) | | Kappa |
| Naïve | Med | 16 (37) | 9 | (50) | 0.118 | 0.882 | 0 | (0) | 18 | (100) | 0 |
| Bayes | Cyto | 26 (62) | 11 | (61) | 0.357 | 0.827 | 6 | (33) | 16 | (89) | 0.222 |
| | Int | 31 (73) | 16 | (89) | 0.653 | 0.986 | 14 | (78) | 18 | (100) | 0.778 |
| Decision | Med | 18 (43) | 6 | (33) | 0.0433 | 0.777 | 12 | (67) | 0 | (0) | 0.333 |
| Tree | Cyto | 27 (64) | 11 | (61) | 0.57 | 0.827 | 6 | (33) | 16 | (89) | 0.222 |
| | Int | 28 (67) | 14 | (78) | 0.524 | 0.936 | 12 | (67) | 16 | (89) | 0.556 |
| Logistic | Med | 11 (27) | 6 | (33) | 0.043 | 0.777 | 6 | (33) | 6 | (33) | 0.333 |
| Regression | Cyto | 31 (73) | 9 | (50) | 0.26 | 0.740 | 10 | (56) | 8 | (44) | 0.000 |
| | Int | 38 (90) | 17 | (94) | 0.727 | 0.999 | 18 | (100) | 16 | (89) | 0.889 |
| Artificial | Med | 17 (40) | 9 | (50) | 0.118 | 0.882 | 6 | (33) | 12 | (67) | 0.000 |
| Neural | Cyto | 33 (79) | 15 | (83) | 0.586 | 0.964 | 14 | (78) | 16 | (89) | 0.667 |
| Network | Int | 38 (90) | 17 | (94) | 0.727 | 0.999 | 16 | (89) | 18 | (100) | 0.889 |

All four supervised machine learning models achieved their highest prediction accuracies when trained with an integrated dataset comprising both patient medical record data and biomaterial-induced blood cytokine levels. This integrated dataset proved particularly effective when utilized in LR and ANN models, achieving 94% (17/18) prediction accuracy. The approach also exceeds predictive healthcare models for other surgical outcomes that utilized only cytokine-based or electronic health record-based approaches. Previous studies demonstrated that their sepsis predictive models had higher predictive power when combined data was utilized, rather than either electronic medical record or biomarker data alone. Interestingly, this integrated approach also identifies new factors of high importance. In contrast to the models trained on cytokine expression alone, models trained with integrated data observed that IL-8 was among the important predictors in DT, LR, and ANN models, and TNF-α in DT and NB models. IL-8 and TNF-α are two prominent pro inflammatory cytokines elevated in patients post-surgery, including mesh implantation and pelvic surgery. In contrast to the models trained on medical record data alone, hemorrhoids were considered one of the important factors in LR and ANN models trained with the integrated dataset. Models trained with the integrated dataset also outperformed those trained with either dataset alone in terms of training accuracy, sensitivity, and specificity across all four models (Table II).

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method of evaluating a subject's risk of one or more complications associated with pelvic organ prolapse surgery, the method comprising:
obtaining, by a computing system comprising one or more computing devices, sample data associated with the subject;
obtaining, by a computing system comprising one or more computing devices, medical record data associated with the subject;
inputting, by the computing system, the sample data and the medical record data into a machine-learned surgical model;
receiving, by the computing system as an output of the machine-learned surgical model, one or more predictions of post-surgical complications of mesh exposure through a vaginal wall associated with the subject; and
performing a pelvic organ prolapse repair surgery on the subject, wherein the surgery is performed based at least in part on the one or more predictions of post-surgical complications by the machine-learned surgical model associated with a likelihood of success.

2. The method of claim 1, wherein the sample data comprises cytokine data.

3. The method of claim 2, wherein the cytokine comprises a pro-inflammatory cytokine, an anti-inflammatory cytokine, a Treg cytokine, a tumor necrosis factor (TNF) superfamily protein, an interferon (IFN) family protein, a T helper 17 (Th17) immunity related gene, a matrix metalloproteinase (MMP), or a combination thereof.

4. The method of claim 2, wherein the cytokine comprises IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-12, IL-29/IFN-11, IL-31, IL-32, IL-33, IL-34, IL-35, sIL-6Ra, pg130/sIL-6Rβ, IFN-a2, IFN-β, IFN-γ, TNF-α, sTNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.

5. The method of claim 1, wherein the medical record data associated with the subject comprises a plurality of the subject's vital sign data, previous diagnoses, social history, or a combination thereof.

6. The method of claim 1, wherein the output comprises an expression visualization that visualizes the predicted post-surgical outcome associated with the subject; and
the method further comprises displaying, by the computing system, the visualization on a display screen.

7. The method of claim 6, further comprising: displaying, by the computing system, the treatment guide on a display screen.

8. The method of claim 1, further comprising: generating, by the computing system based at least in part on the predicted post-surgical outcome output by the machine-learned surgical model, a treatment guide that describes the subject's likelihood of success for the pelvic organ prolapse surgery.

9. A computing system, the computing system comprising:
a machine-learned surgical model trained with training data;
one or more processors; and
one or more non-transitory computer-readable media that store instructions that, when executed by the one or more processors, cause the one or more processors to perform operations, the operations comprising:
obtaining sample data associated with a subject, wherein the data comprises at least one set of sample data associated with the subject;
obtaining medical record data associated with the subject;
inputting the sample data into the machine-learned surgical model; and
receiving, as an output of the machine-learned surgical model, one or more predictions of surgical outcomes associated with the subject.

10. The computing system of claim 9, wherein the sample data comprises a plurality of cytokine marker data.

11. The computing system of claim 10, wherein the cytokine marker data comprises a plurality of cytokine markers present in the data sample of the subject.

12. The computing system of claim 10, wherein the cytokine marker data comprises a pro-inflammatory cytokine, an anti-inflammatory cytokine, a Treg cytokine, a tumor necrosis factor (TNF) superfamily protein, an interferons (IFN) family protein, a T helper 17 (Th17) immunity related gene, a matrix metalloproteinase (MMP), or a combination thereof.

13. The computing system of claim 10, wherein the cytokine marker data comprises IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-12, IL-29/IFN-11, IL-31, IL-32, IL-33, IL-34, IL-35, sIL-6Ra, pg130/sIL-6Rβ, IFN-a2, IFN-β, IFN-γ, TNF-α, STNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.

14. The computing system of claim 9, wherein the sample data associated with the subject comprises a blood sample collected from the subject.

15. The computing system of claim 9, wherein the medical record data associated with the subject comprises a plurality of the subject's vital sign data, previous diagnoses, social history, or a combination thereof.

16. The computing system of claim 9, wherein the training data comprises one or more sets of sample data and medical record data.

17. The computing system of claim 9, wherein:
the output comprises a surgical model visualization that visualizes the one or more expression patterns of cytokine markers associated with the subject; and
the operations further comprise displaying the visualization on a display screen.

18. The computing system of claim 17, wherein the operations further comprise: displaying the treatment guide on a display screen.

19. The computing system of claim 9, wherein the operations further comprise: generating, based at least in part on the predicted post-surgical outcome output by the machine-learned surgical model, a treatment guide that describes the subject's likelihood of success for a pelvic organ prolapse repair surgery.

20. The computing system of claim 9, wherein the machine-learned surgical model comprises one or more of: an artificial neural network, a support vector machine, a decision tree, a naïve bayes, or a logistic regression.
